(19) United States
(12) Patent Application Publication
(10) Pub. No.: US 2024/0090796 A1
(43) Pub. Date: Mar. 21, 2024

(54) Foot Sensor and Analysis Device

(71) Applicant: Decentralized Biotechnology Intelligence Co., Ltd., Taipei City (TW)

(72) Inventors: Yao-Sheng Chou, Taipei City (TW); Hsiao-Yi Lin, Taipei City (TW); Wei-Sheng Su, Taipei City (TW); Hsing-Yu Chi, Taipei City (TW)

(21) Appl. No.: 18/522,896

(22) Filed: Nov. 29, 2023

Related U.S. Application Data
(63) Continuation-in-part of application No. 17/516,635, filed on Nov. 1, 2021.

(30) Foreign Application Priority Data
Nov. 30, 2022 (TW) 111146004

(51) Int. Cl. A61B 5/103; A61B 5/00; A61B 5/11

(52) U.S. Cl. CPC A61B 5/1038; A61B 5/0004; A61B 5/0015; A61B 5/0062; A61B 5/1112; A61B 5/1121; A61B 5/6807; A61B 5/7271; A61B 2560/0214; A61B 2562/0219; A61B 2562/0247; A61B 2562/04; A61B 2562/06

(57) ABSTRACT

A foot sensor and analysis device, which includes a pressure sensing layer arranged inside the insole and a sensing module installed inside the insole. The sensing module is electrically coupled with the pressure sensing layer for receiving and processing detected electronic signals, where sensing module includes an inductance coil to perform wireless charging to the battery. The pressure sensing layer and the sensing module are integrally formed inside the insole.

The present application is a continuation-in-part application of U.S. patent application Ser. No. 17/516,635, filed on Nov. 1, 2021 entitled INSOLE WITH EMBEDDED SENSING SYSTEM, the disclosure of which is hereby incorporated by reference herein.

### TECHNICAL FIELD

The present invention relates to embedded sensor, and more particularly, to a foot sensor and analysis device.

### BACKGROUND

Recently, due to the development of electronic technology, in addition to improvements of accuracy of precision electronic sensors, these sensors have been developed toward to lightweight and portable designs that provide break through beyond the limitations of conventional use, enabling that wearable technologies designed for recording daily physical activities or for professional sports have developed rapidly. However, recently developed functionalities are mainly on simple calculations of step count and stride frequency. In fact, the sensing elements of a wearable device can capture a lot of data during its use. How to further analyze and use these sensed data is the focus of developments in future wearable technology.

Miniaturized electronic sensing components can allow sensors to be installed in shoes, allow experiments to be performed beyond the indoor environment. Also, measurements of the plantar pressure in shoes can also bring additional advantages to motion sensing. Taking running as an example, when the lower limbs are in contact with the ground, despite that instruments such as force plates can accurately measure the external force on the human body, only the plantar pressure can simultaneously provide the time and space parameters of the external force within the plantar area. For example, different forces acting on specific locations of the sole of foot may have different meanings, which may cause the change the probability of sports injuries or falls. The information obtained depends on plantar pressure, which cannot be obtained from other measurement tools. This uniqueness makes the importance of plantar pressure for clinical and sports-related detection cannot be ignored.

The data of plantar pressure distribution can reveal the gait pattern of human body. The measurement of the plantar pressure distribution has great reference value in the fields of biomechanics, rehabilitation medicine, sports training, shoe making and so on. At present, both the clinical pressure test plate and test bench have space limitations and they are both not wearable.

The existing sensors for testing plantar pressure, because its sensing unit is in contact with people's feet, it is easy to wear and tear due to frequent contact with the soles of the feet, which is not conducive to long-term wearing and testing.

Therefore, developing a foot sensor and analysis device, enabling that the sensors can be embedded inside the insole, and the sensor and the insole can be integrally formed during production, can further solve the above deficiencies.

### SUMMARY OF THE INVENTION

The purpose of the present invention is to propose a foot sensor and analysis device enabling that sensors can be embedded in the insole, and the sensor and the insole can be integrally formed during production.

According to one aspect of the present invention, a foot sensor and analysis device is provided, which includes a pressure sensing layer arranged inside the insole and a sensing module installed inside the insole. The sensing module is electrically coupled with the pressure sensing layer for receiving and processing detected electronic signals, where sensing module includes an inductance coil to perform wireless charging to the battery. The pressure sensing layer and the sensing module are integrally formed inside the insole.

In an embodiment, the foot sensor and analysis device further comprises an infrared sensing layer disposed inside said insole and electrically connected to said sensing module to transmit electronic signals detected by said infrared sensing layer, wherein said infrared sensing layer is integrally formed inside said insole.

In an embodiment, the pressure sensing layer includes a plurality of pressure sensors arranged with different density distribution, which are arranged on a forefoot area, a lateral arch area and a heel area in said insole.

In an embodiment, the pressure sensing layer comprises a plurality of resistive pressure sensors.

In an embodiment, the pressure sensing layer is flexible.

In an embodiment, the pressure sensing layer includes a plurality of capacitive sensors with different density distribution, which are arranged on a forefoot area, a lateral arch area and a heel area in said insole.

In an embodiment, the pressure sensing layer is flexible.

In an embodiment, the Infrared sensing layer is flexible.

In an embodiment, the sensing module provides program or algorithm to control collection and storage of data.

In an embodiment, the insole further comprises an accelerometer used to detect direction changes, GPS data, acceleration output data, angular orientation related data, and angular orientation changes during the user's walking, for detecting information including speed/distance, and to correlate with sensed pressure data for cross reference and correction.

According to another aspect of the present invention, a foot sensor and analysis device is provided, which includes a pressure sensing layer arranged inside the insole and a sensing module installed inside the insole. The sensing module is electrically coupled with the pressure sensing layer for receiving and processing detected electronic signals, where sensing module includes an inductance coil to perform wireless charging to the battery. The pressure sensing layer and the sensing module are integrally formed inside the insole. The sensing module includes a processing unit to collect and analyze the electrical signals sensed by the pressure sensing layer and the Infrared sensing layer to convert the electrical signals to a corresponding foot pressure distribution and a blood circulation information, a memory coupled to the processing unit to store the corresponding foot pressure distribution and the blood circulation information, a wireless data transmission/receiving device coupled to the processing unit to transmit said corresponding foot pressure distribution and the blood circulation information to an external electrical device.

In one embodiment, the foot sensor and analysis device further comprises a power supply unit to provide power to said pressure sensing layer, said Infrared sensing layer, said processing unit, said memory and said wireless data transmission/receiving device.

In one embodiment, the pressure sensing layer includes a plurality of pressure sensors arranged with different density distribution, which are arranged on a forefoot area, a lateral arch area and a heel area in said insole.

In one embodiment, said pressure sensing layer comprises a plurality of resistive pressure sensors.

In one embodiment, the pressure sensing layer is flexible.

In one embodiment, the pressure sensing layer includes a plurality of capacitive sensors with different density distribution, which are arranged on a forefoot area, a lateral arch area and a heel area in said insole.

In one embodiment, the sensing module provides program or algorithm to control collection and storage of data.

In one embodiment, the sensing module is configured to communicate with an external electronic device, which is an external computing device, a computing system, a mobile device, or other electronic device type.

In one embodiment, the insole further comprising accelerometer used to detect direction changes, GPS data, acceleration output data, angular orientation related data, and angular orientation changes during the user's walking, for detecting information including speed/distance, and to correlate with sensed pressure data for cross reference and correction.

In one embodiment, the Infrared sensing layer is flexible.

In one embodiment, the wireless data transmission/receiving device is a Bluetooth chip or a WiFi (Wireless Fidelity) device.

### BRIEF DESCRIPTION OF THE DRAWINGS

FIG. 1A shows a top view of an insole with integrated formed sandwich sensors in accordance with one embodiment of the invention.

FIG. 1B shows a side view of an insole with integrated formed sandwich sensors in accordance with one embodiment of the invention.

FIG. 1C shows a distribution diagram of pressure sensors in an insole with integrated formed sandwich sensors in accordance with one embodiment of the invention.

FIG. 1D shows a cross-sectional view of an insole with integrated formed sandwich sensors in accordance with one embodiment of the invention.

FIG. 2 shows a functional block diagram of an insole sensing system in accordance with one embodiment of the invention.

FIG. 3 shows a functional block diagram of a left and a right insole sensing system communicated with an external computing device in accordance with one embodiment of the invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

Plantar pressure is referred to the force per unit area of the human body when the sole of the foot touches the ground during various forms of motion. The plantar pressure detection system includes multiple pressure sensing elements. The plantar pressure parameters can be obtained by the collected and calculated pressure values from each pressure sensing element during the measurement process.

At present, the pressure sensing elements used to detect the plantar pressure can be mainly divided into two types, i.e., capacitive and resistive types of pressure sensing element. The capacitive pressure sensing element uses a diaphragm to separate two conductive plates. When the diaphragm on the sensing element is deformed by pressure, the gap between the diaphragm and the two conductive plates changes, resulting in a change in capacitance, and the magnitude of the pressure can be estimated by measuring the change in capacitance.

The resistive pressure sensing element includes a conductive polymer, and the conductive polymer changes resistance as the pressure changes. The conductive particles of the conductive polymer can be brought into contact by applying force onto, the current through the sensing element is therefore enhanced and the pressure can be calculated.

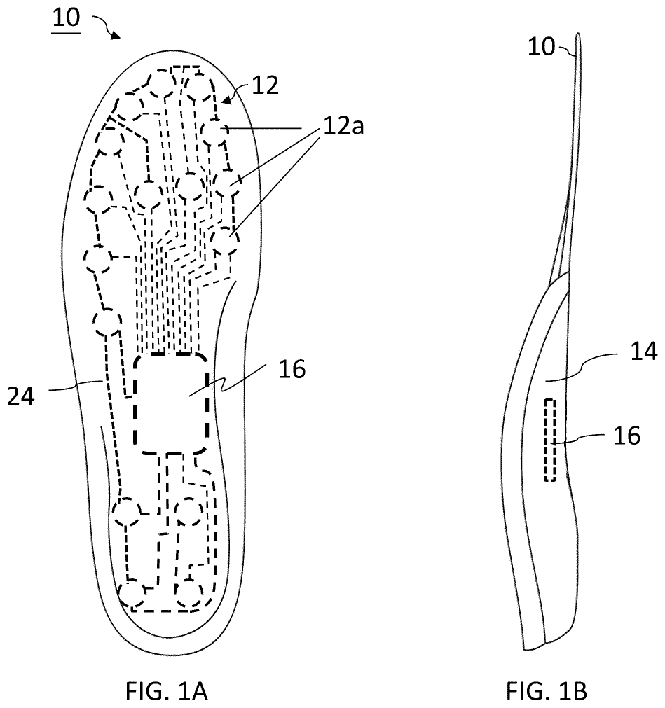

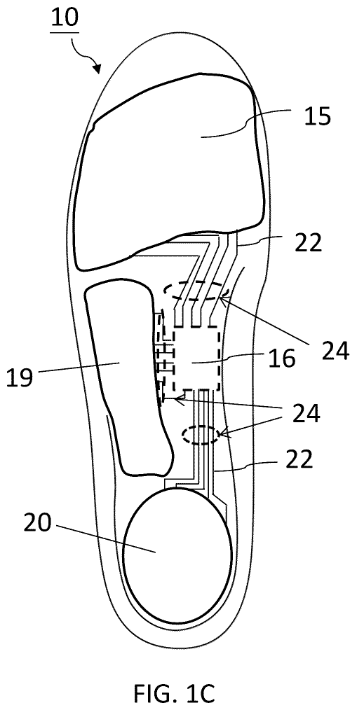

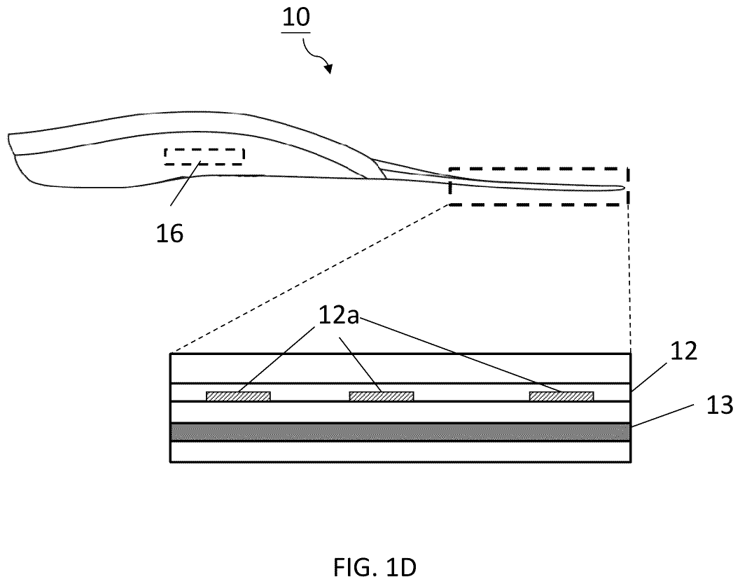

FIG. 1 shows an insole 10 with integrated formed sandwich sensors according to an embodiment of the present invention. FIG. 1A shows a top view of the insole 10. The pressure sensing layer 12 is embedded inside the insole, and illustrates by dashed lines. The pressure sensing layer 12 includes a plurality of pressure sensors 12a to form an array configuration, individual pressure sensors 12a acts as a sensing point to sense the pressure change and position distribution when the foot pressure changes. The pressure sensor 12a is electrically connected to the sensing module 16 through a wire 24.

In a preferred embodiment, the pressure sensor 12a shown in FIG. 1A is a resistive pressure sensing element, which includes a conductive polymer, and the conductive polymer changes resistance as the pressure changes. The conductive particles of the conductive polymer can be brought into contact by applying force onto, the current through the sensing element is therefore enhanced and the pressure can be calculated.

In another preferred embodiment, the pressure sensor 12a shown in FIG. 1A is a capacitive pressure sensor, which includes a diaphragm to separate two conductive plates. When the diaphragm on the sensing element is deformed by pressure, the gap between the diaphragm and the two conductive plates changes, resulting in a change in capacitance, and the magnitude of the pressure can be estimated by measuring the change in capacitance.

FIG. 1B shows a side view of the insole 10. The pressure sensing layer 12 of the insole 10 is integrated formed with sandwich sensors. The insole 10 may include a thermoplastic polyester elastomer (TPEE) layer, and may also include an arch support (cushion) 14 integrated therein. A sensing module 16 is embedded in the arch support 14 of the insole, used to collect, calculate, process and transmit the electronic signals of a plurality of pressure sensors, which can avoid or minimize the contact and stimulation to the wearer's foot.

In an preferred embodiment, the sensing module 16 can be integrated and packaged with a flexible substrate to be disposed in the insole 10 in an integrated molded manner.

As shown in FIG. 1C, the pressure sensors (not shown) in the whole insole can be configured at the positions, such as in the toe area 15, the lateral arch area 19 and the heel area 20, with different density distributions. Each of the pressure sensors is electrically connected to the sensing module 16 by wirings 22. In one embodiment, the pressure sensors with different numbers and corresponding wiring 24 form a flexible pressure sensing device, which is arranged in different parts of the insole to sense the foot pressure distribution of different areas (for example, the positions near the toe area 15, the lateral arch area 19 and the heel area 20) of the user's foot. In other words, the sensing densities of the above three areas are different, in which the density is in order from small to large, i.e., near toe area 15, lateral arch area 19 and heel area 20. Among them, the sensing module 16, including Bluetooth chip and other electronic components, is embedded in the arch support 14 of the arch part of the insole 10. In one embodiment, the sensing module 16 may be an electronic sensing module integrated by a printed circuit board, which has a connection terminal electrically connected with the plurality of pressure sensing devices. The size of the sensing module 16 can be miniaturized, and its thickness is about 3˜4.5 mm (millimeter).

FIG. 1D illustrates a cross-sectional view of the insole 10, and the figure below it is a enlarged partial sectional view of the insole 10 located in the dotted box area. In the figure, individual pressure sensors 12a are arranged in a flexible substrate with corresponding wirings connected, so that individual pressure sensors 12a can transmit the sensed signals to the sensing module 16 through the wirings. In one embodiment, the pressure sensing layer 12 includes a flexible pressure sensor 12a and corresponding wirings.

In addition, the insole 10 can also integrate with an infrared sensor to detect the blood circulation of the user's foot. Based on the high penetration of infrared, the infrared sensor can detect the blood circulation of the foot without clinging to the human skin. FIG. 1D shows that an infrared sensing layer 13 includes a flexible infrared sensor and wiring, which is arranged inside the insole 10 to receive the infrared emitted from the human foot, and this configuration can avoid mutual interference with the pressure sensor. In one embodiment, the infrared sensing layer 13 can be electrically connected with the connection terminal of the sensing module 16 through the flexible wiring. Through the high penetration of the above infrared, the artery on the outside of the instep responsible for delivering blood to the foot is measured, so as to judge whether the instep pulse may be abnormal.

In an embodiment, the pressure sensing layer 12, infrared sensing layer 13 and sensing module 16 can be made flexible, so that the pressure sensing layer 12, the infrared sensing layer 13 and the sensing module 16 can be integrally embedded inside the insole 10 when the insole 10 is injection molded. Among them, the insole 10 adopts the wireless charging mode, the overall insole can be completely free of exposed holes, and the insole 10 can be cleaned in a washing machine.

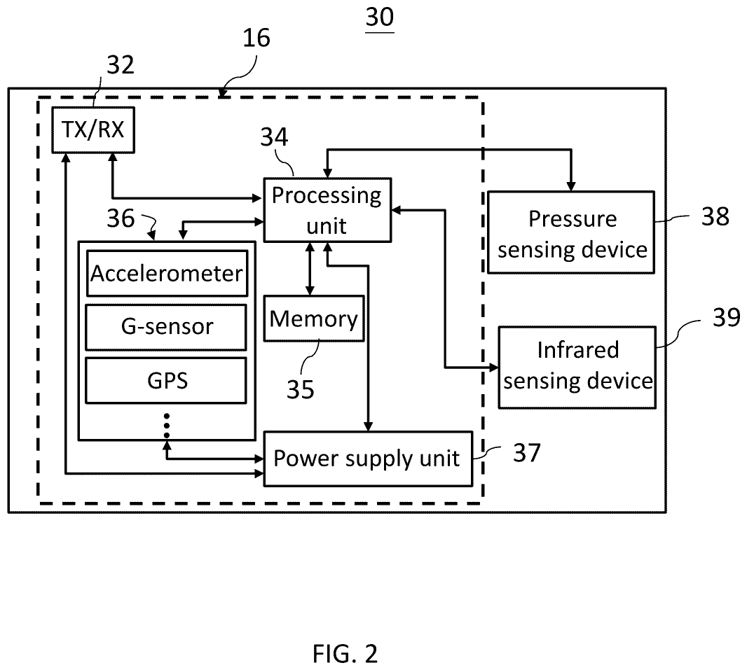

FIG. 2 shows a functional block diagram of an insole sensing system 30, which includes a sensing module 16 to perform data transmitting/receiving through a wireless data transmission/receiving device (TX/RX) 32. As depicted in FIG. 2, the wireless data transmission/receiving device (TX/RX) 32 is integrated into the sensing module 16, those skilled in the art can understand that the wireless data transmission/receiving device (TX/RX) 32 can also be a separate component for the purpose of data transmission/receiving. In the example of FIG. 2, the sensing module 16 includes a wireless data transmission/receiving device (TX/RX) 32 for transmitting data to and/or receiving data from one or more remote systems. In one embodiment, the wireless data transmission/receiving device (TX/RX) 32 may be a Bluetooth chip, WiFi (Wireless Fidelity) or similar wireless data transmission/receiving device. The sensing module 16 can be electrically connected with a plurality of pressure sensors (pressure sensing device 38) and an infrared sensing device 39 arranged in the insole via a connection terminal. The sensing module 16 also includes a processing unit 34 (such as, one or more microprocessors), a memory 35, additional sensors 36 (including accelerometer, gyroscope (G-sensor), global positioning system (GPS) sensor, etc.) 36, and a power supply unit 37. The power supply module 37 can provide electrical power to the pressure sensing device, infrared device 39 and/or other components of the sensing system (for example, processing unit 34, memory 34, additional sensors 36). In one preferred embodiment, the power supply module 37 includes a rechargeable solid-state battery, an induction coil (for coupling with an external wireless charging system to charge the battery wirelessly) and a USB charging interface. It should be realized that the sensing module 16 can provide computer programs/algorithms to control the collection and storage of data (for example, user's foot pressure distribution data or pressure data interacting with the ground, user's foot blood cycle state, etc.), and these programs/algorithms can be stored and/or executed.

The TX/RX device 32 can connect to one or more sensors, and transmit or provide the detection data or information related to various different parameters created by the additional sensors 36. These data or information include physiological data related to the user, speed data/distance information of pedometer type. The accelerator is used to detect change of directions during walking detected by accelerometer, GPS data, acceleration output/data, angular orientation related data and change of angular orientation (sensing by G-sensor), and these data can be stored in the memory or transmitted to a remote computing device or server via the TX/RX device 32.

In the embodiment of FIG. 2, the sensing module 16 may include a startup system (not shown). That is, the system or a part thereof can be coupled to the sensing module 16 or connected to the insole or separated from other parts of the sensing module 16. The startup system may be used to selectively start the sensing module 16. In one embodiment, the sensing module 16 may be operated through a specified mode to enable the sensing module to be started or closed, such as a pressure threshold applied to one or more sensors. In other embodiments, the sensing module 16 may be turned on or off through a button. In any of these embodiments, the sensing module 16 may include a sleep mode that may put the system into the sleep mode after a period of inactive state. In one embodiment, for example, the G-sensor does not detect activity within a preset time, and the sensing module 16 may enter sleep mode to save power.

The sensing module 16 may also be configured to communicate with an external device, which may be an external computing device, a computing system, a mobile device (smart phone, tablet, etc.), or other electronic devices.

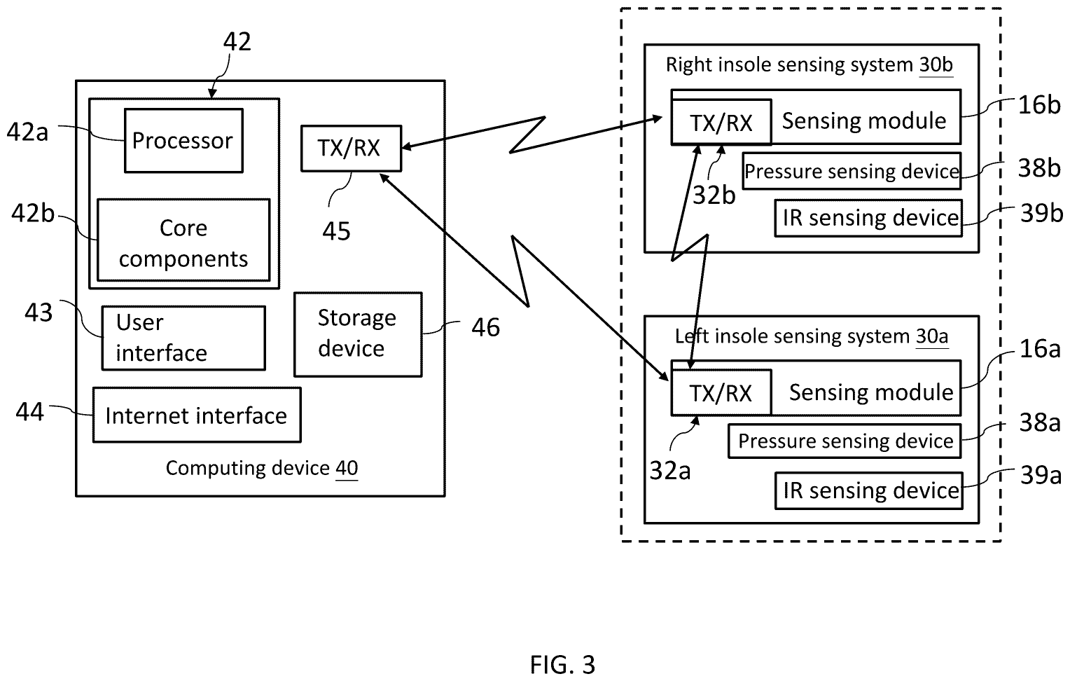

FIG. 3 shows a system block diagram of communication between the left and right insole sensing systems (30a, 30b) and an external computing device 40. The left and right insole sensing systems (30a, 30b) each includes a sensing module (16a, 16b) embedded in the arch of the insole, which is electrically connected with a pressure sensing devices (38a, 38b) and an infrared sensing device (39a, 39b) to receive and analyze a pressure distribution of the user's foot and a blood circulation data of foot, and transmit the above data to a remote computing device or server through a TX/RX device (32a, 32b) located in the sensing module. The sensing modules (16a, 16b) each include a processing unit (such as, one or more microprocessors), a memory, additional sensors, and a power supply unit (referring to FIG. 2).

The external computing device 40 is any electronic device that can transmit data, process data, and/or store data. In one embodiment, the computing device 40 is a portable computing device and/or a fixed computing device. The portable computing device may be a social network device, a game device, a mobile phone, a smart phone, a personal digital assistant, a digital audio/video player, a notebook computer, a tablet computer, a video game controller, and/or any other portable device containing a computing core. The fixed computing device may be a personal computer (PC), a computer server, a television, a printer, a fax machine, a home entertainment device, a video game console, and/or any type of home or office computing device containing a computing core.

The external computing device 40 includes a computing core 42, a user interface 43, an Internet interface 44, a wireless communication transceiver 45, and a storage device 46. The user interface 43 includes one or more input devices (such as, keyboard, touch screen, voice input device, etc.), one or more audio output devices (such as, speaker, headphone jack, etc.), and/or one or more video output devices (such as, video graphics display, touch screen, etc.). The Internet interface 44 includes one or more networking devices (such as, wireless local area network (WLAN) devices, wired LAN devices, wireless wide area network (WWAN) devices, etc.). The storage device 46 includes a flash memory device, one or more hard disk drives, one or more solid-state (SS) storage devices, and/or a cloud memory.

The computing core 42 includes a processor 42a and other computing core components 42b. Other computing core components 42b include a video graphics processing unit, a memory controller, a main memory (such as RAM), one or more input/output (I/O) device interface modules, input/output (I/O) interfaces, input/output (I/O) controllers, peripheral device interfaces, one or more USB interface modules, one or more network interface modules, one or more memory interface modules and/or one or more peripheral device interface modules.

The wireless communication transceiver 45 of the external computing device 40 and the wireless data transmission/receiving devices (32a, 32b) of the insole sensing system 30 have similar transceiver types (such as, Bluetooth, WLAN, WiFi, etc.). The wireless data transmission/receiving devices (32a, 32b) communicate directly with the wireless communication transceiver 45 to share the collected data and/or receive instructions from the external computing device 40 through the respective insole sensing system 30. In addition or as an alternative example, the wireless data transmission/receiving devices (32a, 32b) communicate with one of them to collect data. The wireless data transmission/receiving device 32a transmits the collective data to the wireless communication transceiver 45 of the external computing device 40.

The external computing device 40 processes data to produce various results. For example, the external computing device 40 processes the data from the sensing system 16 in combination with the circuit of algorithm, which can analyze any data related to foot pressure during movement, such as the pressure distribution on the wearer's left and right feet, the ratio of weight to the left and right feet, gait, gait frequency, and the center of pressure (COP) during body dynamics.

Foot pressure distribution plays a critical role in a movement of human body. A posture of human body and changes in the bone are affected by foot shape and walking (running) posture, which also affects the performance and limit in sports. The invention proposes an insole with integrated formed sandwich sensors, which can obtain the parameter data of foot pressure distribution of many users for time and space through the insole arranged in the shoe, and upload the data to external computing devices (e.g. smart phone, personal computer, computer servers, etc.) to calculate, analyze and store the data in the cloud system as relevant database of big data.

In addition, the insole with integrated formed sandwich sensors can also integrate an infrared detection device to synchronously provide the user's blood circulation information. Breaking through the limitation that only medical institutions or sports research institutions can obtain data analysis in the past, the invention can facilitate more sports and more users obtaining exclusive movement or motion analysis. Synchronously, it also enables the establishment and use of data platforms in various professional fields, so that different professionals (such as sports, health care, shoemaking, etc.) can establish their linkage relationship with foot pressure performance.

In one embodiment, the above-mentioned data is transmitted wirelessly, while combined with the APP, it can be displayed in real time, so that the above-mentioned data can be visualized.

The above-mentioned data collected by the insole with integrated formed sandwich sensors can be applied in more diverse sports and lets more users to obtain exclusive personal movement or sports analysis. Simultaneously, it also opens up the establishment and use of data platforms in various professional areas, so that different professional sports can establish a linkage between sport and foot pressure performance, and further develop various algorithms and apps, thereby unlocking the mysteries of human movement and posture.

In addition, the sensing insole proposed by the present invention adopts a wireless charging mode, the entire insole can be completely free of exposed holes, and the insole can be washed in a washing machine; moreover, the sensing module and the insole can be in integrally formed during injection molding.

While various embodiments of the present invention have been described above, it should be understood that they have been presented by a way of example and not limitation. Numerous modifications and variations within the scope of the invention are possible. The present invention should only be defined in accordance with the following claims and their equivalents.

What is claimed is:

1. A foot sensor and analysis device, comprising:
a pressure sensing layer arranged inside said insole;
a sensing module installed inside said insole, said sensing module electrically coupled with said pressure sensing layer for receiving and processing detected electronic signals;
wherein said sensing module includes an inductance coil to perform wireless charging to a battery of said sensing module;
wherein said pressure sensing layer and said sensing module are integrally formed inside said insole.

2. The device of claim 1, further comprising an infrared sensing layer disposed inside said insole and electrically connected to said sensing module to transmit electronic signals detected by said infrared sensing layer, wherein said infrared sensing layer is integrally formed inside said insole.

3. The device of claim 1, wherein said pressure sensing layer includes a plurality of pressure sensors arranged with different density distribution, which are arranged on a forefoot area, a lateral arch area and a heel area in said insole.

4. The device of claim 3, wherein said pressure sensing layer comprises a plurality of resistive pressure sensors.

5. The device of claim 4, wherein said pressure sensing layer is flexible.

6. The device of claim 1, wherein said pressure sensing layer includes a plurality of capacitive sensors with different density distribution, which are arranged on a forefoot area, a lateral arch area and a heel area in said insole.

7. The device of claim 6, wherein said pressure sensing layer is flexible.

8. The device of claim 2, wherein said Infrared sensing layer is flexible.

9. The device of claim 1, wherein said sensing module provides program or algorithm to control collection and storage of data.

10. The device of claim 9, further comprising an accelerometer used to detect direction changes, GPS data, acceleration output data, angular orientation related data, and angular orientation changes during the user's walking, for detecting information including speed/distance, and to correlate with sensed pressure data for cross reference and correction.

11. A foot sensor and analysis device, comprising:
a pressure sensing layer arranged inside said insole;
an infrared sensing layer configured within said insole;
a sensing module, configured in an arch support integrated with said insole, coupled to said pressure sensing layer and said infrared sensing layer for receiving and processing electronic signals sensed by said pressure sensing layer and said infrared sensing layer;
wherein said pressure sensing layer, said infrared layer and said sensing module are integrally formed inside said insole;
wherein said sensing module includes:
a processing unit to collect and analyze said electrical signals sensed by said pressure sensing layer and said Infrared sensing layer to convert said electrical signals to a corresponding foot pressure distribution and a blood circulation information;
a memory coupled to said processing unit to store said corresponding foot pressure distribution and said blood circulation information;
a wireless data transmission/receiving device coupled to said processing unit to transmit said corresponding foot pressure distribution and said blood circulation information to an external electrical device.

12. The device of claim 11, further comprising a power supply unit to provide power to said pressure sensing layer, said Infrared sensing layer, said processing unit, said memory and said wireless data transmission/receiving device.

13. The device of claim 11, wherein said pressure sensing layer includes a plurality of pressure sensors arranged with different density distribution, which are arranged on a forefoot area, a lateral arch area and a heel area in said insole.

14. The device of claim 13, wherein said pressure sensing layer comprises a plurality of resistive pressure sensors.

15. The device of claim 14, wherein said pressure sensing layer is flexible.

16. The device of claim 11, wherein said pressure sensing layer includes a plurality of capacitive sensors with different density distribution, which are arranged on a forefoot area, a lateral arch area and a heel area in said insole.

17. The device of claim 16, wherein said pressure sensing layer is flexible.

18. The device of claim 11, further comprising accelerometer used to detect direction changes, GPS data, acceleration output data, angular orientation related data, and angular orientation changes during the user's walking, for detecting information including speed/distance, and to correlate with sensed pressure data for cross reference and correction.

19. The device of claim 11, wherein said Infrared sensing layer is flexible.

20. The device of claim 11, wherein said wireless data transmission/receiving device is a Bluetooth chip or a WiFi (Wireless Fidelity) device.
